(19) United States
(12) Patent Application Publication
(10) Pub. No.: US 2021/0307769 A1
(43) Pub. Date: Oct. 7, 2021

(54) SYSTEMS AND METHODS THAT INCREASE THE EFFICACY OF MAGNETIC RESONANCE GUIDED FOCUSED ULTRASOUND (MRgFUS) APPLICATIONS

(71) Applicant: Vanderbilt University, Nashville, TN (US)

(72) Inventors: Sandeep S. ARORA, Nashville, TN (US); Charles F. CASKEY, Nashville, TN (US); William A. GRISSOM, Nashville, TN (US)

(21) Appl. No.: 16/322,673

(22) PCT Filed: Aug. 4, 2017
(86) PCT No.: PCT/US2017/045502

§ 371 Date: Feb. 1, 2019

Related U.S. Application Data
(60) Provisional application No. 62/371,386, filed on Aug. 5, 2016.

(51) Int. Cl. A61B 17/22; A61B 8/08; G16H 20/30; G01R 33/48; G01R 33/56

(52) U.S. Cl. CPC A61B 17/22004; A61B 8/481; A61B 8/085; A61B 2017/320069; G01R 33/4804; G01R 33/4814; G01R 33/5601; G16H 20/30

(57) ABSTRACT

Applications related to non-invasive magnetic resonance guided focused ultrasound (MRgFUS) in a patient's vasculature are described. For example, the applications can include an ablation procedure, an occlusion procedure, a cauterization procedure, and the like. Accordingly, one aspect of the present disclosure is directed to a method for performing an MRgFUS application that includes selecting a target area within a patient's vasculature, configuring multifocal acoustic waves, and applying the multifocal acoustic waves to the target area to heat sequential locations in the target area simultaneously to facilitate the MRgFUS application.

### CROSS-REFERENCE TO RELATED APPLICATIONS

This application claims the benefit of U.S. Provisional Application No. 62/371,386, entitled “Systems and Methods for Increasing Efficacy of Magnetic Resonance Guided Focused Ultrasound (MRgFUS) Ablation,” filed Aug. 5, 2016. The entirety of this provisional application is hereby incorporated by reference for all purposes.

### TECHNICAL FIELD

The present disclosure relates generally to magnetic resonance-guided focused ultrasound (MRgFUS) and, more particularly, to systems and methods that increase the efficacy of MRgFUS applications.

### BACKGROUND

Magnetic resonance-guided focused ultrasound (MRgFUS) is a non-invasive treatment technique that combines focused ultrasound and magnetic resonance imaging (MRI). Applying a focused ultrasound beam to a target tissue causes a rapid local increase in temperature within the target tissue. The associated MRI can provide on-line thermometric imaging to allow for real-time adjustment of the treatment parameters based on the local temperature increase. MRgFUS has been used in connection with conditions as varied as treatment of uterine fibroids, palliation of pain, cancer tissue ablation, and essential tremor treatment. While MRgFUS is an attractive non-invasive treatment option for many medical conditions, MRgFUS is not widely used for applications in a patient's vasculature due to limitations related to procedure time, respiratory motion, and the heat sink effect.

### Summary

The present disclosure relates generally to magnetic resonance-guided focused ultrasound (MRgFUS) and, more particularly, to systems and methods that increase the efficacy of MRgFUS applications. For example, when the MRgFUS application is targeted to an area in a patient's vasculature, the systems and methods of the present disclosure can reduce limitations related to procedure time, respiratory motion, and the heat sink effect.

In one aspect, the present disclosure can include a method for increasing the efficacy of non-invasive MRgFUS applications (e.g., an ablation procedure, an occlusion procedure, a sono-cauterization procedure, etc.). The method can include steps performed by a system comprising a processor, including: selecting a target area within a patient's vasculature for application of the MRgFUS and configuring a plurality of multifocal acoustic waveforms to be applied to the target area. An acoustic delivery device can apply the multifocal acoustic waves to the target area to heat sequential locations in the target area simultaneously to facilitate the application of the MRgFUS. In some instances, the MRgFUS can occur with an accelerant so that the thermal delivery can be amplified based on the accelerant, which enables rapid and accurate thermal dose delivery to the target area for the application.

In another aspect, the present disclosure can include a system that increases the efficacy of a non-invasive MRgFUS application (e.g., an ablation procedure, an occlusion procedure, a sono-cauterization procedure, etc.). The system can include a computing device comprising a non-transitory memory storing instructions and a processor to execute the instructions which includes selecting a set of sequential locations in a target area of a patient's vasculature for the MRgFUS application and configuring a set of multifocal acoustic waves to apply to the set of sequential locations simultaneously. The system can also include an acoustic source to apply the set of multifocal acoustic waves to the target area in order to heat the sequential locations in the target area simultaneously, facilitating the MRgFUS application in the target area. In some instances, the system can include an intravenous delivery device to deliver an accelerant to the patient's vasculature. The accelerant can amplify the thermal delivery to enable rapid and accurate thermal dose delivery to the target area.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is an illustration of an example system for non-invasive magnetic resonance guided focused ultrasound (MRgFUS) applications in accordance with an aspect of the present disclosure;

FIG. 2 is an illustration of an example of a MRgFUS scanner that can be used with the system of FIG. 1;

FIG. 3 is a process flow diagram showing a method for non-invasive MRgFUS applications in accordance with another aspect of the present disclosure;

FIG. 4 is a process flow diagram showing another method for MRgFUS applications that includes MR thermometry feedback;

FIG. 5 is a process flow diagram showing another method for MRgFUS applications that includes the addition of an accelerant;

FIG. 6 is a schematic representation of an example of multifocal targeting of acoustic focal areas within a target area (a portion of a large vessel) with an arrow signifying the direction of blood flow;

FIG. 7 is an experimentally obtained MR thermometry image of a line of heat created over a 2-cm length in pork demonstrating the feasibility of this approach; and

FIG. 8 shows various illustrations, images, and plots showing multi-focal heating in an experimental flow channel.

### DETAILED DESCRIPTION

### I. Definitions

Unless otherwise defined, all technical terms used herein have the same meaning as commonly understood by one of ordinary skill in the art to which the present disclosure pertains.

In the context of the present disclosure, the singular forms “a,” “an” and “the” can also include the plural forms, unless the context clearly indicates otherwise

As used herein, “comprises” and/or “comprising” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “magnetic resonance-guided focused ultrasound” or “MRgFUS” can refer to a treatment technique that combines focused ultrasound and magnetic resonance imaging. In some instances, MRgFUS can provide a non-invasive treatment modality for an MRgFUS application.

As used herein, the term “MRgFUS application” can include a medical use of MRgFUS. In some instances, the MRgFUS application can be medical procedure involving a patient's vasculature, like an ablation procedure, an occlusion procedure, a sono-cauterization procedure, or the like.

As used herein, the term “ultrasound” can refer to acoustic waves having an ultrasonic frequency. For example, the ultrasonic frequency can be a frequency used for high intensity focused ultrasound (HIFU) (e.g., lower than medical diagnostic ultrasound, from 0.250 MHz to 2 MHz, but providing significantly higher energy than medical diagnostic ultrasound).

As used herein, the term “acoustic waves” can refer to mechanical and longitudinal waves that result from an oscillation of pressure that travels through a solid, liquid, or gas in a wave pattern.

As used herein, the term “focused” ultrasound can refer to multiple intersecting beams of ultrasound concentrated on one or more points within a target area. The multiple intersecting beams can be concentrated on the target, in some instances, by an acoustic lens. The focused application of acoustic waves can be referred to as sonication.

As used herein, the term “magnetic resonance imaging” or “MRI” can refer to the use of a magnetic field and pulses of radio wave energy to generate images of organs and structures within the body.

As used herein, the term “thermometric imaging” can refer to the use of MRI for creation of images mapping the temperature distribution of a target area. With thermometric imaging, one or more thermometry maps can be created of at least a portion of a target area based on proton resonance frequency shifts,

As used herein, the term “target area” can refer to an area that the MRgFUS application is performed on or within. The target area can include, but is not limited to, areas within a tissue, organ, or the vasculature.

As used herein, the term “accelerant” can refer to any material that can be injected intravenously to improve the speed and/or increase the efficacy of MRgFUS applications. Accelerants can include, but are not limited to, phase shift nanodroplets (PSND), microbubbles, and iron nanoparticles.

As used herein, the term “efficacy” can refer to the ability to produce a desired or intended result. As an example, the efficacy of an MRgFUS application can be improved by increasing the speed of a procedure associated with the MRgFUS application. As another example, the efficacy of an MRgFUS application can be improved by limiting the effects of respiratory motion and/or the heat sink effect.

As used herein, the terms “disease” and “condition” can refer to any disorder of structure or function in a patient.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### II. Overview

The present disclosure relates generally to magnetic resonance-guided focused ultrasound (MRgFUS). MRgFUS is an attractive non-invasive treatment option for many medical conditions. MRgFUS is an appealing treatment option at least because MRgFUS is superior to current invasive methods of treatment as MRgFUS does not result in the negative side effects associated with current invasive treatment methods. The superiority of MRgFUS over invasive treatment methods has led to use of MRgFUS in connection with conditions as varied as treatment of uterine fibroids, palliation of pain, cancer tissue ablation, and essential tremor treatment. Additionally, MRgFUS has been proposed as a potential non-invasive treatment for vascular conditions requiring applications like ablation, occlusion, sono-cauterization, or the like. Examples of such vascular conditions include, but are not limited to, treatment of vascular malformations, hemorrhage control, and tumor devascularization. However, currently MRgFUS is not widely used for such vascular applications due to limitations related to the generally long procedure time, the effects of respiratory motion, and/or the heat sink effect. The present disclosure relates, more specifically, to systems and methods that can improve the efficacy of MRgFUS when used for vascular applications. However, the systems and methods described herein can be used for MRgFUS in connection with any use limited by the time associated with the procedure, respiratory motion, and/or the heat sink effect (e.g., abdominal applications).

### III. Systems

One aspect of the present disclosure can include a system 1, shown in FIG. 1, for non-invasive MRgFUS applications. For example, the MRgFUS applications can be vascular applications (including ablation procedures, occlusion procedures, sono-cauterization procedures, and the like), but need not be limited to vascular applications. Indeed, the system 1 can be used in connection with any use limited by the time associated with the procedure, respiratory motion, and/or the heat sink effect (e.g., abdominal applications). The system 1 can increase the efficacy of these MRgFUS applications. For example, the system 1 can improve the speed (and, therefore, reduce treatment time) associated with MRgFUS applications. As another example, the system 1 can counteract the effects of respiratory motion and/or the heat sink effect.

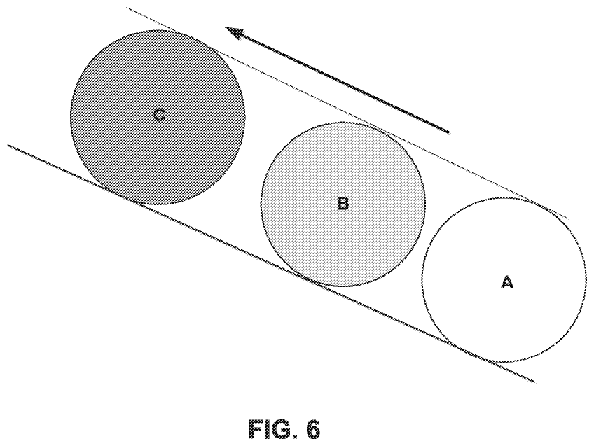

The system 1 can use multi-focal targeting to heat a set of sequential locations in one or more target areas related to a disease under treatment simultaneously or sequentially to facilitate the MRgFUS application, while improving the efficacy of the MRgFUS application. However, in each target area, the set of sequential locations can be heated simultaneously (see, e.g., FIG. 6, showing a set of sequential locations A, B, and C in a target volume of a patient's blood vessel). As used herein, the term “sequential” when used with “locations” can refer to areas located distally from each other in the vascular system in a direction of blood flow. For example, referring to FIG. 6, locations A, B, and C are sequential locations, where location A is upstream from location B, which is upstream from location C. In FIG. 6, the sequential locations A, B, and C are arranged linearly. However, the sequential locations need not be arranged linearly depending on the shape of the target area. For example, by heating the upstream locations (A, B) and downstream location (C) simultaneously, the heat deposition can be increased and the heat sink is reduced at the downstream location (C) to cause collagen fusion and stable vascular occlusion

The system 1 can include a computing device 2 and an acoustic source 8. The computing device 2 can select 6 the set of sequential locations in the target area and configure 7 a set of multifocal acoustic waves to apply to the set of sequential locations simultaneously. The acoustic source 8 can apply the set of multifocal acoustic waveforms to the target area to heat the sequential locations in the target area simultaneously to facilitate the MRgFUS application.

The computing device 2 can include an input/output (I/O) component 3, a non-transitory memory 5 and one or more processors 4. The I/O component 3 can be a hardware device that allows communication via the computing device 2 and one or more external devices, like the acoustic source 8. The I/O component 3 can also facilitate data entry (e.g., though a keyboard, a mouse, a touch screen, or the like) or display (e.g., on a monitor or other display device, printed by a printer, or the like)

In some instances, the non-transitory memory 5 and the one or more processors 4 can be hardware devices. Software aspects that can be implemented by the computing device 2 can be stored as computer program instructions in the non-transitory memory 5. The non-transitory memory 5 can be any non-transitory medium that can contain or store the computer program instructions, including, but not limited to, a portable computer diskette; a random-access memory; a read-only memory; an erasable programmable read-only memory (or Flash memory); and a portable compact disc read-only memory). The computer program instructions may be executed by the one or more processors 4. The one or more processors 4 can be one or more processors of a general purpose computer, special purpose computer, and/or other programmable data processing apparatus.

The one or more processors 4 can execute instructions from the non-transitory memory 5 to select 6 the set of sequential locations in the target area and configure 7 the set of multifocal acoustic waves to apply to the set of sequential locations simultaneously. The set of sequential locations can be a plurality of focus points arranged linearly within the target area (shown, for example, as locations A, B, C in the vasculature in FIG. 6). The acoustic waves can be configured such that heat gathers and is held within one or more of the focus points.

The acoustic source 8 can receive instructions from the computing device 2 (e.g., over a wired connection, a wireless connection, or a combination thereof) to apply the set of multifocal acoustic waves to the target area to heat the sequential locations in the target area simultaneously to facilitate the MRgFUS application. Using the example of FIG. 6, each of locations A, B, and C can be heated with the multifocal acoustic waves simultaneously. The intensity of heating at each location can be same or varied. As the blood flows from location A to location C, the blood can be heated at each of locations A, B, and C. Therefore, the blood in location C can have a higher temperature (accrued from locations A and B, as well as heated at location C) than either of location A or location B. The heat at location C can facilitate applications, including an ablation procedure, an occlusion procedure, a sono-cauterization procedure, or the like.

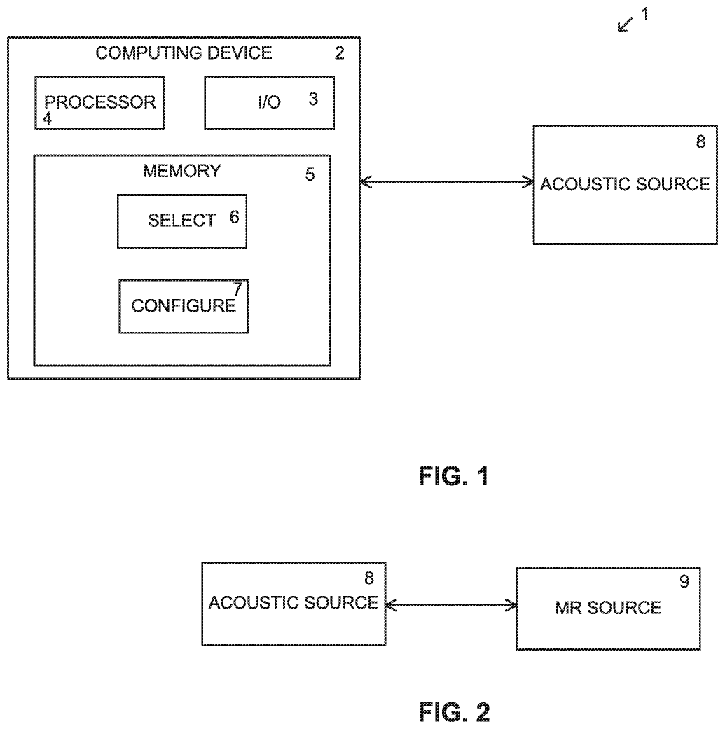

In some instances, as shown schematically in FIG. 2, the acoustic source 8 can be part of a clinical MRgFUS scanner. The clinical MRgFUS scanner can include a magnetic resonance (MR) source 9 in addition to the acoustic source 8. The MR source 9 can image the target area using MRI to acquire MR images. The acquired MR images can quantify a temperature distribution over time using MR thermometry maps created from proton resonance frequency shifts. In some instances, a magnetic resonance (MR) contrast agent or an ultrasound contrast agent can be introduced intravenously into the patient's vasculature to aid in determination of completion of vascular occlusion. If occlusion is incomplete, more sonications can be performed.

Based on data reflecting the temperature distribution recorded, the amplitude of the multifocal acoustic waves can be varied over time. The MRgFUS scanner can be in communication with the computing device 2 for instructions related to the variation in the amplitude of the acoustic waves in response to the feedback from the MR source 9. However, in some instances, the MRgFUS scanner can be equipped with a processor (e.g., a microprocessor) to perform the adjustment of the amplitude of the acoustic waves.

In some instances, the system 1 can also include an intravenous access device to administer an accelerant to the patient intravenously. The efficacy of heating the sequential locations in the target area is increased based on the presence of the accelerant. In some instances, the accelerant confines the heating to the sequential locations without damaging near field healthy tissues outside of an acoustic focus. For example, the multifocal acoustic waves can provide a sonic pressure to the accelerant to trigger a phase change from the liquid to a gas. The phased changed accelerant can be acoustically active and can confine the heating to the sequential locations without damaging near field healthy tissues outside of the acoustic focus.

The accelerant can include as one or more phase shift nanodroplets (PSND), microbubbles, or iron nanoparticles, to reduce the time required for image-guided treatment with MRgFUS dramatically. The accelerant can be intravenously administered to a patient as a liquid. In some instances, the accelerant can have a low boiling point so that the liquid accelerant can phase shift to a gas within the vasculature when exposed to the appropriate threshold pressure. For example, the multifocal acoustic waves can provide a sonic pressure to the accelerant to trigger a phase change from the liquid to a gas. The phased changed accelerant can be acoustically active and can confine the heating to the sequential locations without damaging near field healthy tissues outside of the acoustic focus. Because these accelerants are only activated at the acoustic focus in the region to be ablated, acoustic effects are confined to the diseased region, sparing healthy tissue. This formulation avoids the major challenge in using traditional microbubbles as thermal delivery amplifiers, which is damage to healthy tissues in the near field and outside of the acoustic focus.

### IV. Methods

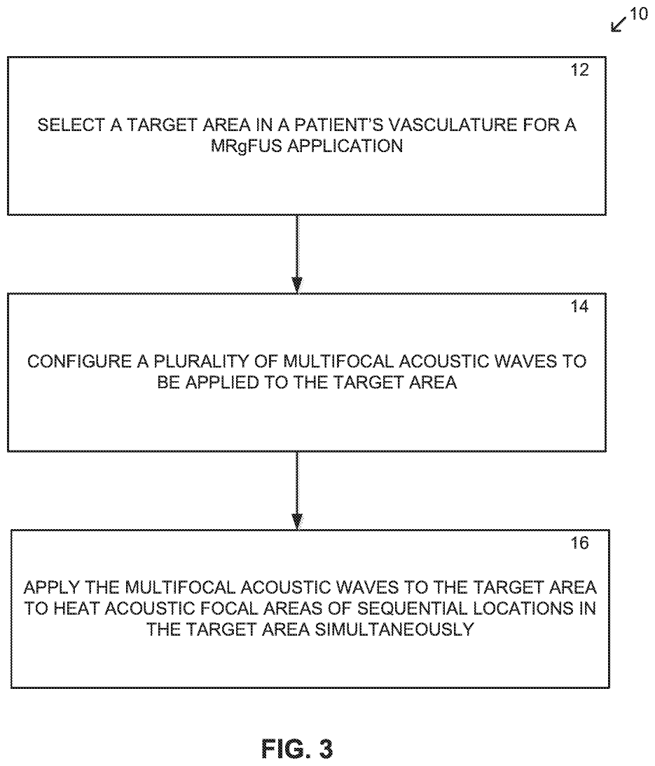

Another aspect of the present disclosure can include methods 10, 20, 30 for non-invasive MRgFUS applications, as shown FIGS. 3-5. For example, the MRgFUS applications can be vascular applications (including ablation procedures, occlusion procedures, sono-cauterization procedures, and the like), but need not be limited to vascular applications.

The methods 10, 20, 30 are each illustrated as a process flow diagram with a flowchart illustration. For purposes of simplicity, the method 10 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 10. Moreover, various hardware devices (e.g., the computing device 2 and the acoustic source 8 of the system 1) can be utilized to execute the methods 10, 20, and 30. Additionally, methods 10, 20, 30 are not exclusive of each other. For example, one or more of methods 10, 20, and 30 can be performed together.

FIG. 3 illustrates a method 10 for non-invasive MRgFUS applications. The method 10 improves the efficacy of MRgFUS treatments, which opens new applications for MRgFUS therapy. The method 10 enables accurate targeting of one or more elements (e.g., the vasculature) related to the disease under treatment. The method 10 can also use multi-focal targeting (for example, to heat a volume along the length of a vessel of interest). The method 10 is especially useful in applications suffering from the effects of respiratory motion and/or beset by the heat sink effect (e.g., flowing blood dissipates heat).

At Step 12, a target area in a patient's vasculature can be selected for an MRgFUS application. The selection can be accomplished by a computing device comprising a memory and a processor (like the computing device 2 of FIG. 1). For example, the target area can include multiple focus points arranged linearly within the target area.

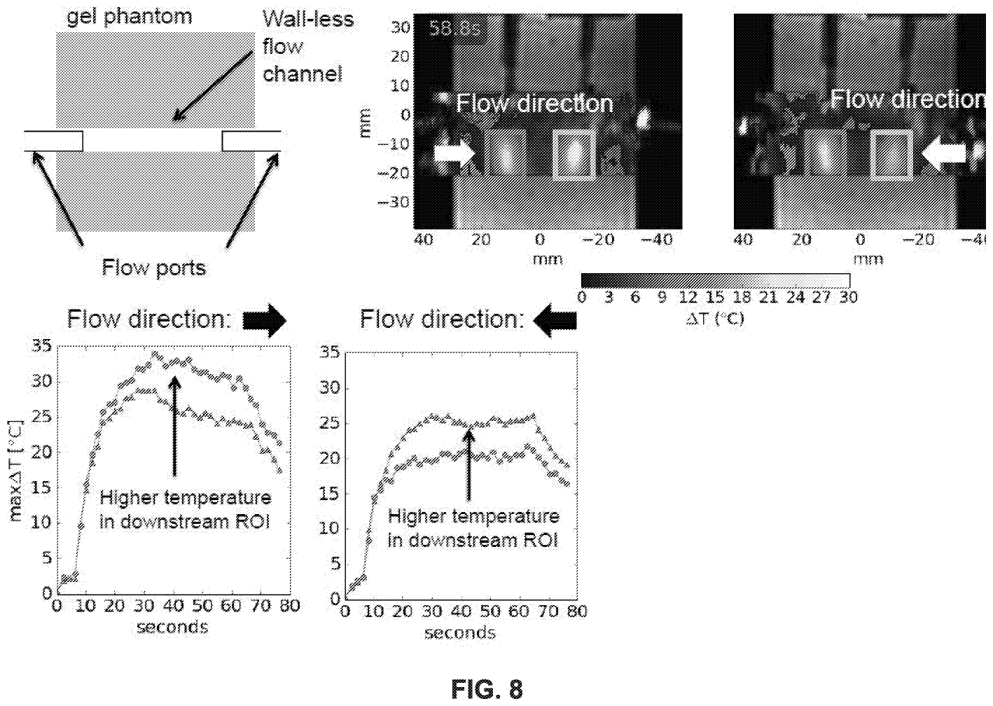

At Step 14, a plurality of multifocal acoustic waves can be configured to be applied to the target area. At step 16, multifocal acoustic waves can be applied to the target area of the patient to heat sequential locations in the target area simultaneously. For example, the sequential locations can correspond to the multiple focus points (e.g., locations A, B, and C, as shown in FIG. 6). For example, the multiple focus points can be arranged linearly. However, the multiple focal points need not be arranged linearly and, instead, can be arranged in any type of shape that is dictated by the shape of the target area. The heating of the sequential locations allows a volume of blood to remain in the heated region longer, resulting in a successful heating at the most downstream location due to additive heat and decreased flow (shown, for example, in FIGS. 6 and 8)

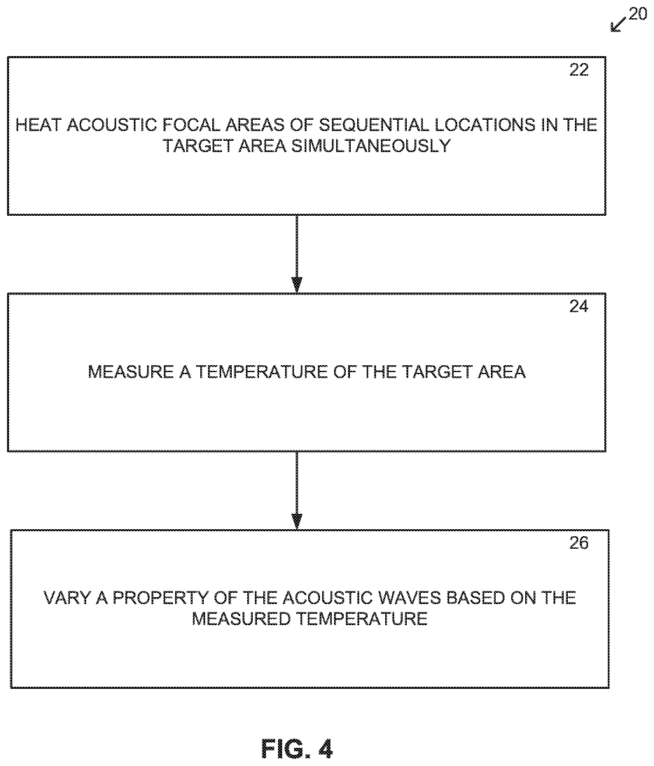

The multifocal acoustic waves can be delivered by a clinical MRgFUS scanner to control the multifocal acoustic waves based on acquired MR images. FIG. 4 illustrates another method 20 for MRgFUS applications that includes MR thermometry feedback. At Step 22, the acoustic focal areas of the sequential locations in the target area can be heated simultaneously. At Step 24, the temperature of the target area can be measured or determined. For example, the MRgFUS device can acquire one or more MR images of the target area. For example, the acquired MR images can quantify a temperature distribution over time using MR thermometry maps created from proton resonance frequency shifts At Step 26, a property of the acoustic waves can be varied based on the measured temperature. For example, a phase and/or an amplitude of the multifocal acoustic waves can be varied based on data shown in the MR thermometry maps. In some instances, a magnetic resonance (MR) or ultrasound contrast agent can be introduced into the patient's vasculature to aid in determination of completion of the MRgFUS application (e.g., occlusion, ablation, or sono-cauterization).

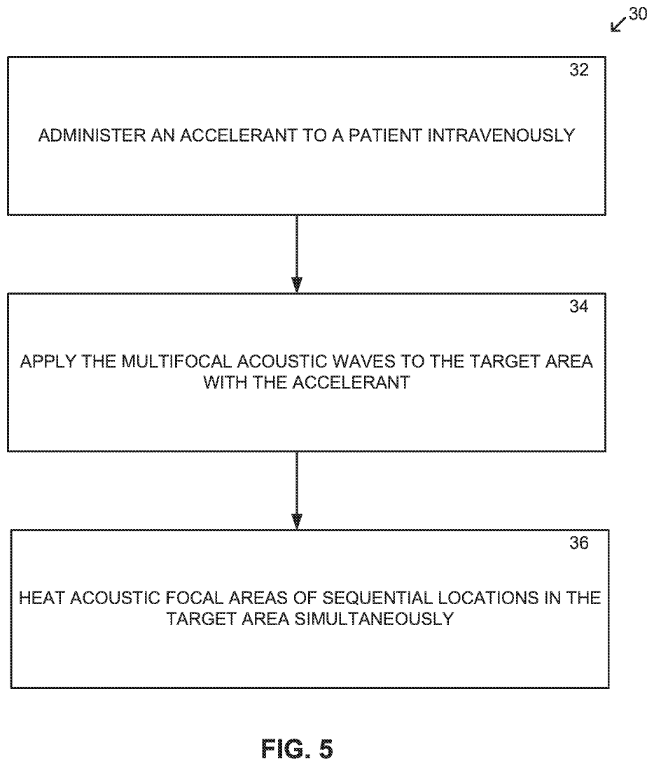

Multi-focal targeting can be used in conjunction with an accelerant, such as one or more phase shift nanodroplets (PSND), microbubbles, or iron nanoparticles, to dramatically reduce the time required for image-guided treatment with MRgFUS. FIG. 5 illustrates another method 30 for MRgFUS applications that includes the addition of an accelerant. At Step 32, an accelerant can be administered to the patient intravenously. At Step 34, the multifocal acoustic waves can be applied to the target area with the accelerant. At Step 36, acoustic focal areas of sequential locations in the target area can be heated sequentially. The accelerant can expedite the heating.

The accelerant can be intravenously administered to a patient as a liquid. In some instances, the accelerant can have a low boiling point so that the liquid accelerant can phase shift to a gas within the vasculature when exposed to the appropriate threshold pressure. For example, the multifocal acoustic waves can provide a sonic pressure to the accelerant to trigger a phase change from the liquid to a gas. The phased changed accelerant can be acoustically active and can confine the heating to the sequential locations without damaging near field healthy tissues outside of the acoustic focus. Because these accelerants are only activated at the acoustic focus in the region to be ablated, acoustic effects are confined to the diseased region, sparing healthy tissue. This formulation avoids the major challenge in using traditional microbubbles as thermal delivery amplifiers, which is damage to healthy tissues in the near field and outside of the acoustic focus.

In some instances, the accelerant can be a PSND that can include liquid encapsulated low boiling point perfluorocarbons at a ratio tailored to a certain acoustic threshold for undergoing the phase change. For example, the PSND can include a fluorocarbon element, a lipid element, and a buffer element. More specifically, the PSND can include lipid encapsulation and low boiling point perfluorocarbons that enable the production of metastable 100-300 nm droplets that are stable in liquid form and can be formulated with the same excipients used in other FDA approved ultrasound contrast agents. Upon exposure to a specific acoustic threshold, which can be tailored based on the perfluorocarbon ratio in the core, the PSNDs convert to microbubbles of approximately 5 fold increase in diameter (e.g., 1 micron bubble from a 200 nm droplet). The size range of the PSND may enable them to stay in vessels and extravasate in leaky tumor vasculature, further increasing the selectivity of ablation in tumor applications. Accordingly, the application of the PSNDs can allow for predictable sonication and therapeutic margins with substantially lower acoustic energy delivery.

Indeed, the use of multifocal imaging in combination with PSND administration can open new applications of MRgFUS therapy that are not currently feasible with traditional MRgFUS therapy, providing a significant advancement for the future of focused ultrasound therapies. The use of multifocal targeting can allow heating of a volume along a length of a certain area of interest (e.g., a certain vessel), which can increase the heating effects down the line. The use of the accelerants can amplify heating effects in the certain area of interest. This strategy will benefit all applications of MRgFUS, but especially those beset by the heat-sink effect (highly vascularized areas, like the liver or vessels themselves, are affected at least because flowing blood dissipates heat). As PSNDs are only activated at the acoustic focus in the region to be ablated, acoustic effects are confined to the diseased region, sparing healthy tissue. This formulation avoids damaging healthy tissues in the near field and outside of the acoustic focus, which has presented the major challenge in using traditional microbubbles as thermal delivery amplifiers.

### V. Experimental

The following example is for the purpose of illustration only and is not intended to limit the scope of the appended claims. Instead, the following example merely shows the feasibility of using non-invasive multi-focal heating to improve the efficacy of MRgFUS applications in a patient's vasculature. For example, the multifocal targeting (which can be aided by the administration of an accelerant) has been shown to (a) lead to complete occlusion of flow in lobar portal veins, (b) lead to faster ablation of VX2 liver tumor implants, and (c) lead to sono-cauterization of superficial femoral arteries in a rabbit model.

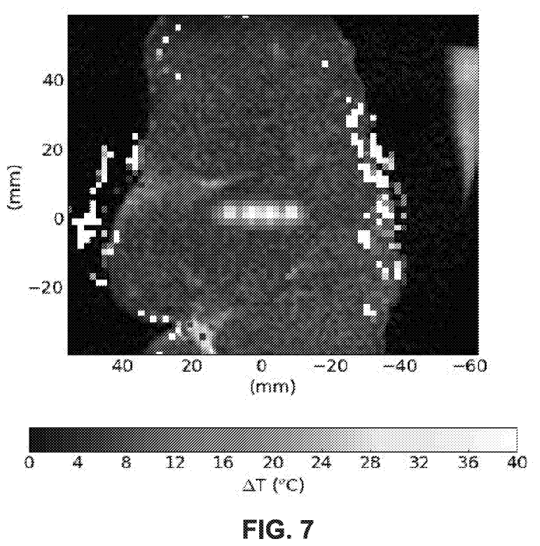

Multi-focal insonation was used to show that heat sink effects can be reduced by heating along a treated vessel, thereby increasing the length of time that a volume of blood is within the acoustic focus. Multi-focal patterns capable of heating a single line were programmed using the MatHIFU, the freely available toolbox for Matlab hosted at github as a private hub repository freely available to researchers under coordination of Philips Healthcare and executed with the Philips Sonalleve (Philips Healthcare, Andover, Mass.). As demonstrated in the thermometry image of FIG. 7, the multi-focal patterns are capable of creating a line of heat over 2 cm in length in pork.

A test of multi-focal heating of a vessel is shown in FIG. 8. The vessel was modeled by generating a gel phantom with tissue-like thermal characteristics (2% agar, 1% graphite) and a wall-less flow channel with −3 mm diameter (FIG. 8, top left). A peristaltic pump flowed water through the vessel at a rate of 15 cm/s, and heating was examined with MR thermometry during a sonication with two simultaneous foci (center frequency of 1.6 MHz, power of 80W). MR thermometry images were acquired during sonication with two foci and flow in the forward (left) and reverse (right) directions (FIG. 8, top right, boxes indicate regions of interest (ROI) for temperature measurement). Notably, a higher temperature in the downstream side regardless of flow direction was observed (FIG. 8, bottom, showing that the maximum temperature occurs in the downstream ROI, regardless of flow direction), demonstrating that heat is absorbed when the fluid passes the first sonication point and is further heated at the second location.

From the above description, those skilled in the art will perceive improvements, changes and modifications. Such improvements, changes and modifications are within the skill of one in the art and are intended to be covered by the appended claims.

The following is claimed:

1. A method comprising:
selecting, by a system comprising a processor, a target area in a patient's vasculature for a magnetic resonance guided focused ultrasound (MRgFUS) application;
configuring, by the system, a plurality of multifocal acoustic waves to be applied to the target area;
applying, by an acoustic delivery device, the multifocal acoustic waves to the target area, wherein the multifocal acoustic waves heat acoustic focal areas of sequential locations in the target area simultaneously to facilitate the MRgFUS application,
wherein the MRgFUS application is at least one of an ablation procedure, an occlusion procedure, and a sono-cauterization procedure.

2. The method of claim 1, further comprising administering an accelerant to the patient intravenously; and
increasing an efficacy of the heating the sequential locations based on a presence of the accelerant.

3. The method of claim 2, wherein the accelerant comprises at least one of a plurality of phase shift nanodroplets (PSNDs) and a plurality of microbubbles.

4. The method of claim 2, wherein the accelerant is configured to undergo a phase change from liquid to gas upon exposure to the multifocal acoustic waves.

5. The method of claim 4, wherein the multifocal acoustic waves provide a sonic pressure to the accelerant to trigger the phase change.

6. The method of claim 4, wherein the accelerant comprises a low boiling point per fluorocarbon at a ratio tailored to a certain acoustic threshold for undergoing the phase change.

7. The method of claim 2, wherein the accelerant confines the heating to the sequential locations without damaging near field healthy tissues outside of the acoustic focus.

8. The method of claim 1, wherein the acoustic delivery device comprises a clinical magnetic resonance guided focused ultrasound (MRgFUS) scanner to control application of the multifocal acoustic waves based on acquired MR images.

9. The method of claim 8, wherein the acquired MR images quantify a temperature distribution over time using MR thermometry maps created from proton resonance frequency shifts

10. The method of claim 9, wherein at least one of a phase or an amplitude of the multifocal acoustic waves is varied based on data shown in the MR thermometry maps.

11. The method of claim 8, further comprising introducing a magnetic resonance (MR) or ultrasound contrast agent into the patient's vasculature to aid in determination of complete vascular occlusion.

12. The method of claim 1, wherein the heating of the acoustic focal areas of the sequential locations are heated simultaneously to allow a volume of blood to remain in a downstream portion of the target area due to additive heat and decreased flow.

13. The method of claim 1, wherein the sequential locations are arranged linearly.

14. A system comprising:
a computing device comprising:
a memory storing instructions; and
a processor to execute the instructions to at least:
select a set of sequential locations in a target area of a patient's vasculature for a magnetic resonance guided focused ultrasound (MRgFUS) application; and
configure a set of multifocal acoustic waves to apply to the set of sequential locations simultaneously; and
an acoustic source to apply the set of multifocal acoustic waves to the target area to heat the sequential locations in the target area simultaneously to facilitate the MRgFUS application,
wherein the MRgFUS application is at least one of an ablation procedure, an occlusion procedure, and a sono-cauterization procedure.

15. The system of claim 14, further comprising an intravenous access device to administer an accelerant to the patient intravenously,
wherein accelerant increases an efficacy of the heating the sequential locations based on a presence of the accelerant.

16. The system of claim 15, wherein the accelerant confines the heating to the sequential locations without damaging near field healthy tissues outside of an acoustic focus.

17. The system of claim 14, wherein the acoustic source comprises a clinical magnetic resonance guided focused ultrasound (MRgFUS) scanner,
wherein the processor executes the instructions to acquire MR images; and control the multifocal acoustic waves based on acquired MR images.

18. The system of claim 17, wherein the acquired MR images quantify a temperature distribution over time using MR thermometry maps created from proton resonance frequency shifts.

19. The system of claim 18, wherein at least one of a phase or an amplitude of the multifocal acoustic waves are varied based on data shown in the MR thermometry maps.

20. The system of claim 14, wherein the sequential locations are arranged linearly.
